(19) United States
(12) Patent Application Publication
(10) Pub. No.: US 2010/0183699 A1
(43) Pub. Date: Jul. 22, 2010

(54) COMPOSITIONS AND METHODS TO CROSS LINK POLYMER FIBERS

(76) Inventors: Wankei Wan, London (CA); Mina Mekhail, Montreal (CA)

Correspondence Address: MILLER THOMSON LLP, 100 STONE ROAD WEST, SUITE 301, GUELPH, ON, N1G-5L3, CA

(21) Appl. No.: 12/691,020

(22) Filed: Jan. 21, 2010

Related U.S. Application Data
(60) Provisional application No. 61/117,997, filed on Jan. 21, 2009.

(51) Int. Cl. C07D 311/02; C09K 3/00; C08L 77/00; C07K 1/00; C07H 1/00; C08K 5/1545; C12N 5/00; A61K 38/17; A61F 2/02; A61K 38/39; A61P 17/00

(52) U.S. Cl. 424/423; 549/396; 252/380; 252/182.24; 525/434; 530/410; 536/123.1; 524/110; 530/350; 435/399; 514/12

(57) ABSTRACT

Novel compositions comprising genipin for cross-linking polymer fibers, are provided. In aspects of the invention the compositions further comprise a solvent system, wherein said solvent system comprises alcohol solvent and water. The genipin-based compositions are useful in methods for promoting the stabilization of fibers in an aqueous environment, and in tissue engineering. The novel genipin-based composition is also useful in methods of treating dermatological conditions.

### FIELD OF THE INVENTION

The present invention relates to novel methods and compositions for cross-linking and stabilizing fibers in aqueous environments and to the fibers treated with said compositions. More particularly, the present invention relates to compositions comprising genipin and to methods of treating fibers having a primary amine group with the compositions of the invention to prevent, ameliorate and/or reduce destabilization of the fibers in an aqueous environment. Fibers treated in accordance with the methods of the present invention are useful in tissue engineering, controlled release/drug delivery, wound healing, cosmetic applications and other biomedical applications.

### BACKGROUND OF THE INVENTION

Tissue engineering is a new cross-disciplinary field between bioengineering, life sciences and clinical sciences to solve critical medical problems related to tissue loss and organ failure by using synthetic or naturally derived, engineered biomaterials to replace damaged or defective tissues, such as bone, skin, and even organs.

A major challenge in tissue engineering is the design of ideal scaffolds that can mimic the structure and biological functions of the natural extracellular matrix. As such, the biomaterial of choice must be biocompatible, biodegradable (with no cytotoxic by-products) and allow cellular attachment, migration and proliferation. In addition the biomaterial should provide physical support to the cells as remodelling takes place. Furthermore, the scaffold must be stable in an aqueous environment such as that provided in the extracellular matrix. One biomaterial that satisfies all of the previously mentioned requirements is collagen, which is a fibrous structural protein that is abundant in the body and is responsible for mechanical strength in tissues. Collagen has been known to self assemble to form a protein scaffold that can be used to structurally support cell or tissue proliferation and various techniques for fabricating collagen scaffolds have been disclosed.

Previous attempts that used collagen nanofibers manufactured by electrospinning methods have proven to be possible, but the resulting fibers are inherently unstable in an aqueous environment (Matthews, J. A., et al., Biomacromolecules 2002, 3, (2), 232-8; Rho, K. S., et al., Biomaterials 2006, 27, (8), 1452-61; Zhong, S., et al., Biomacromolecules 2005, 6, (6), 2998-3004; Zhong, S., et al., Biomed Mater Res A 2006, 79, (3), 456-63; and Yang, L., et al., Biomaterials 2008, 29, (8), 955-962). Protein nanofibers tend to undergo significant swelling and eventually lose their fiber structure and mechanical integrity. FIG. 1A is a scanning electron microscope (SEM) image showing the typical as-spun nanofibers. The non-woven architecture shown, together with the porosity and pore interconnectivity are essential for tissue engineering scaffolds. The fiber size and size distribution histogram of the as spun collagen fibers are also shown in FIG. 1B. These fibers are stable in air. However, upon contact with water, they rapidly swell and disintegrate thus losing their nanofibrous morphology. FIG. 2 is an SEM image of collagen fibers that have been exposed to water for five minutes; the nanofibrous structure is lost and there is no discernable structure on the sub-micrometer scale. It is therefore necessary to explore approaches that would allow the maintenance and control of fiber morphology.

One approach to enhance physical and chemical stability of protein fibers in an aqueous environment is by chemical crosslinking. Glutaraldehyde (GA) vapour has been extensively used to crosslink electrospun collagen nanofibers. This approach, however, has proven to be rather ineffective since most of the GA crosslinked fibers swell significantly in water and form gel-like structures even after exposure to GA vapor over extended periods of time (Rho, K. S., et al., Biomaterials 2006, 27, (8), 1452-61). Furthermore, GA has also been shown to be highly cytotoxic to cells when released from the crosslinked samples over time (Gendler, E., et al., J Biomed Mater Res 1984, 18, (7), 727-36; Gough, J. E., et al., Biomed Mater Res 2002, 61, (1), 121-30; Huang-Lee, L. L., et al., Biomed Mater Res 1990, 24, (9), 1185-201; and Marinucci, L., et al., Biomed Mater Res A 2003, 67, (2), 504-9). Alternatives to GA such as 1-ethyl-3-(3-dimethylaminopropyl) carbodiimide (EDC) and N-hydroxysuccinimide (NHS) result in fibers with significant degree of swelling and loss in both the nanofibrous morphology and porosity (Barnes, C. P., et al., Tissue Engineering 2007, 13, (7), 1593-1605).

Genipin is a natural compound that is derived from geniposide, an iridoid glycoside found in the fruits of Gardenia jasminoides Ellis. The geniposide is isolated, purified and hydrolyzed with B-glucosidase to produce genipin. Genipin is a naturally occurring cross-linker to fix biological tissue.

U.S. Pat. Appl. No. 20080195230 discloses the use of genipin to fix whole, natural tissues to reduce the antigenicity and immunogenicity and prevent enzymatic degradation of the tissue when implanted in a host. Cross linking whole tissues, however, results in shrinking of the tissue thereby affecting and preventing cellular attachment, migration and proliferation therein.

It would be desirable, thus, to develop an alternative method of producing a polymer fiber that is stable in an aqueous environment and is suitable in industrial and biomedical applications, which overcomes at least one of the disadvantages of the current fibers and manufacturing methods.

### SUMMARY OF THE INVENTION

The Applicants have identified novel compositions comprising genipin for improving the stability of fibers in an aqueous environment. The Applicant has demonstrated that a polymer fiber cross linked with the novel compositions of the present invention can be stable in aqueous environments and is suitable for industrial and biomedical applications.

As such, the present invention encompasses the novel composition comprising genipin in a variety of methods, uses and applications, including industrial and biomedical applications.

Thus, in one aspect the present invention provides for a composition for cross-linking fibers, characterized in that said composition comprises genipin.

In another aspect, the present invention provides for a composition useful for promoting the stabilization of fibers in an aqueous environment, characterized in that said composition comprises genipin in an amount effective to prevent, ameliorate and/or reduce destabilization of the fibers in the aqueous environment.

In aspects, the compositions of the invention further comprise a solvent system.

In aspects of the invention, the solvent system comprises a solvent and water.

In aspects of the invention, the water in the solvent system is present in an amount from about 1 v/v % to about 5 v/v % of the total solvent.

In aspects of the invention, the solvent is an alcohol.

In aspects of the invention, the alcohol in the solvent system is selected from the group consisting of ethanol and isopropanol.

In aspects, the compositions of the invention comprise no less than about 0.5 wt % of genipin.

In aspects of the invention, the fibers comprise continuous nanofibers.

In aspects of the invention, the fibers are selected from the group comprising of: collagen, elastin, aminopolysaccharides, gelatin, silk, fibrin, laminin and polyamides.

In aspects of the invention, the aqueous environment comprises an extra-cellular matrix.

In a further aspect, the present invention provides for a composition for cross-linking continuous nanofibers, characterized in that said composition comprises genipin, an alcohol solvent and water, wherein said genipin, alcohol, and water are provided in an amount effective to prevent, ameliorate and/or reduce destabilization of the continuous nanofibers in an aqueous environment.

In another aspect, the present invention provides for a method of cross-linking fibers, characterized in that said method comprises the step of contacting the fibers with a composition comprising genipin.

In another aspect, the present invention provides for a method of promoting the stabilization of fibers in an aqueous environment, characterized in that said method comprises the step of contacting the fibers with a composition comprising genipin in an amount effective to prevent, ameliorate and/or reduce destabilization of the fiber in the aqueous environment.

In aspects of the present invention, the methods are characterized in that said composition further comprises a solvent system.

In aspects of the present invention, the methods are characterized in that the solvent system comprises a solvent and water.

In aspects of the present invention, the methods are characterized in that the water in the solvent system is present in an amount from about 0.1 v/v % to about 5 v/v % of the total solvent.

In aspects of the present invention, the methods are characterized in that the solvent is an alcohol.

In aspects of the present invention, the methods are characterized in that the alcohol in the solvent system is selected from the group consisting of ethanol and isopropanol.

In aspects, the compositions of the invention comprise no less than about 0.5 wt % of genipin.

In aspects of the present invention, the methods are characterized in that each fiber comprises a continuous nanofiber.

In aspects of the present invention, the methods are characterized in that the fibers are selected from the group comprising of: collagen, elastin, aminopolysaccharides, gelatin, silk, fibrin, laminin and polyamides.

In aspects of the present invention, the methods are characterized in that the aqueous environment comprises an extra-cellular matrix.

In another aspect, the present invention provides for a method of controlling the degree of swelling of a fiber in an aqueous environment, characterized in that said method comprises contacting the fiber with a composition comprising genipin, an alcohol and water for a time of treatment, wherein the degree of swelling is controlled by selecting the amounts of genipin, alcohol or water in the composition, or by selecting the time of treatment.

In another aspect, the present invention provides for a fiber, characterized in that said fiber has been treated in any of a composition comprising genipin in an amount effective to prevent, ameliorate and/or reduce destabilization of the fiber in an aqueous solution.

In another aspect, the present invention provides for a scaffold comprises fibers treated in a composition comprising genipin in an amount effective to prevent, ameliorate and/or reduce destabilization of the fibers in an aqueous environment. In aspects of the invention, the scaffold further comprises at least one cell.

In another aspect, the present invention provides for a method of preparing nanofibrous scaffolds for use in tissue regeneration/engineering, said method comprising the following steps: (a) producing nanofibers; (b) treating the nanofibers with a composition comprising genipin, alcohol and water, and wherein said genipin, alcohol solvent and water are present in an amount effective to prevent, ameliorate and/or reduce destabilization of the nanofibers in an aqueous environment.

In another aspect, the present invention provides for a method of treating a dermatological condition comprising the step of topically applying to the skin or lip a collagen fiber treated with a composition comprising genipin, alcohol and water, wherein said genipin, alcohol and water are present in an amount effective to prevent, ameliorate and/or reduce destabilization of the collagen fiber in an aqueous environment.

In another aspect, the present invention provides for a device for the controlled release of a pharmaceutically active agent, said device comprising: (a) a fiber matrix, wherein the fiber in the matrix includes a primary amine group and the polymer fiber is treated with a composition comprising genipin, alcohol and water, wherein said genipin, alcohol and water are present in an amount effective to prevent, ameliorate and/or reduce destabilization of the collagen fiber in an aqueous environment; and (b) a pharmaceutically active agent, wherein said pharmaceutically active agent is incorporated in the polymer fiber matrix.

Advantages of the present invention include the production of fibers, including proteinaceous biodegradable fibers such as collagen nanofibers, that: (1) are more stable (retain their morphology and three-dimensional structure) in aqueous environments, (2) are less cytotoxic, (3) result in a more effective control of fiber swelling and (4) do not include irregularities such as beading or gel-like bodies.

These and other aspects of the invention will become apparent from the detailed description that follows, and the following figures in which:

### BRIEF DESCRIPTION OF THE DRAWINGS

FIG. 1A illustrates a SEM image of as-spun collagen nanofibers;

FIG. 1B is a histogram representing as-spun collagen fiber diameter distribution;

FIG. 2 is a SEM image of uncrosslinked collagen fibers;

FIG. 3 is a SEM image of collagen nanofibers exposed to water after being crosslinked in a solution comprising [A] genipin and absolute ethanol solution and [B] genipin and absolute isopropanol;

FIG. 4 illustrates [A] as-spun collagen nanofiber material [B] collagen nanofibers after genipin-crosslinking using four crosslinking conditions;

FIG. 5 illustrates SEM images of collagen fibers crosslinked using the four conditions of FIG. 4;

FIG. 6 graphically illustrates average collagen fiber diameters after exposure to DMEM;

FIG. 7 graphically illustrates degree of crosslinking of collagen fibers;

FIG. 8 illustrates a calibration curve for the ninhydrin assay; and

FIG. 9 illustrates the chemical structure of genipin.

### DETAILED DESCRIPTION OF THE INVENTION

### i. Definitions

For convenience, the meaning of certain terms and phrases employed in the specification, examples, and appended claims, are provided below. Unless defined otherwise, all technical and scientific terms used herein have the same meaning as commonly understood by one of ordinary skill in the art to which this invention belongs. Also, unless indicated otherwise, except within the claims, the use of “or” includes “and” and vice-versa. Non-limiting terms are not to be construed as limiting unless expressly stated or the context clearly indicates otherwise (for example “including”, “having” and “comprising” typically indicate “including without limitation). Singular forms including in the claims such as “a”, “an” and “the” include the plural reference unless expressly stated otherwise.

“Alcohol” is used herein to denote any organic compound in which a hydroxyl group (—OH) is bound to a carbon atom of an alkyl or substituted alkyl group. The general formula for a simple acyclic alcohol is CₙH₂ₙ₊₁OH. Examples of an alcohol include ethanol, isopropanol, methanol, propanol, n-butanol, sec-butanol, isobutanol and ter-butanol.

“Drug”, “therapeutic agent”, “therapeutic” and the like indicates any molecule that has a significant effect on the body to treat or prevent conditions or diseases.

“Fiber” as used herein is meant to refer to continuous polymer fibers, including micro and nanofibers, having a primary amine group and that find applications in tissue engineering and biomedical fields. Examples of polymer fibers include: collagen, elastin, chitosan (aminopolysaccharides), gelatin, silk, fibrin, laminin and polyamides.

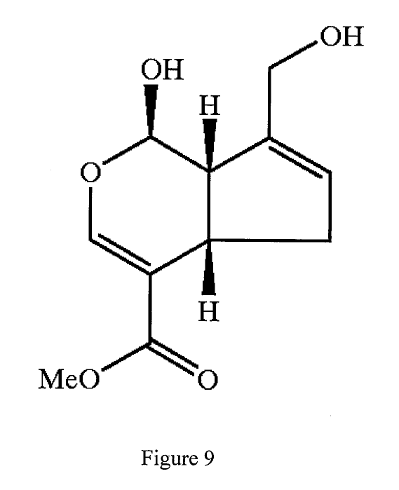

“Genipin” refers to a naturally occurring compound shown in FIG. 9 and to its stereoisomers and mixtures thereof. Genipin is a natural compound that is derived from geniposide, an iridoid glycoside found in the fruits of Gardenia jasminoides Ellis. The geniposide is isolated, purified and hydrolyzed with B-glucosidase to produce genipin.

“Pharmaceutically active agent” means any of a drug, therapeutic agent, pro-drug or diagnostic.

“Polymer” indicates a molecule composed of a number of repeat units.

### ii. Controlling the Stability of Fibers in an Aqueous Environment

The present invention provides for a composition for cross-linking fibers, wherein said composition comprises genipin and for methods for cross-linking fibers with a composition comprising genipin.

The Applicants have developed and identified novel compositions that specifically interact with fibers leading the development of fibers that are capable of retaining their morphology and three-dimensional structure in an aqueous environment. Thus, the present invention has several industrial applications such as in the fabrication of fiber-based tissue engineering scaffold having controlled swelling and degradation rate. The present invention also has several biomedical applications such as the controlled release of pharmaceutically active agents and other compounds, wound healing, treatment of dermatological conditions.

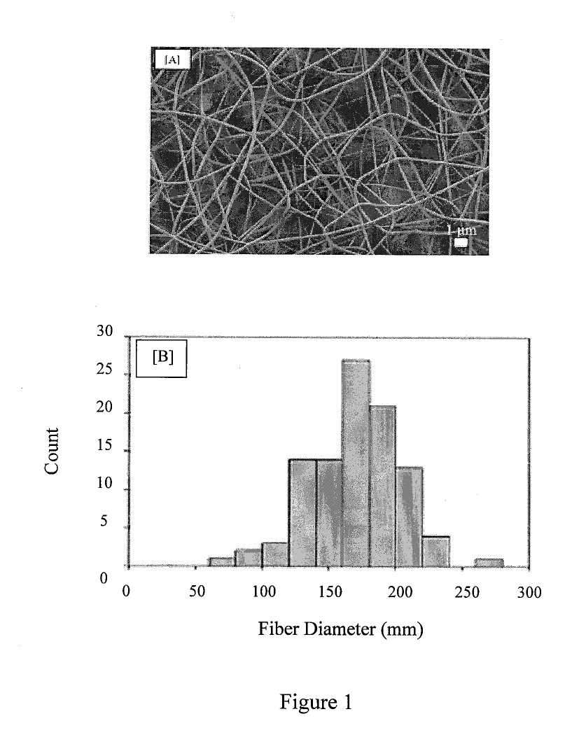

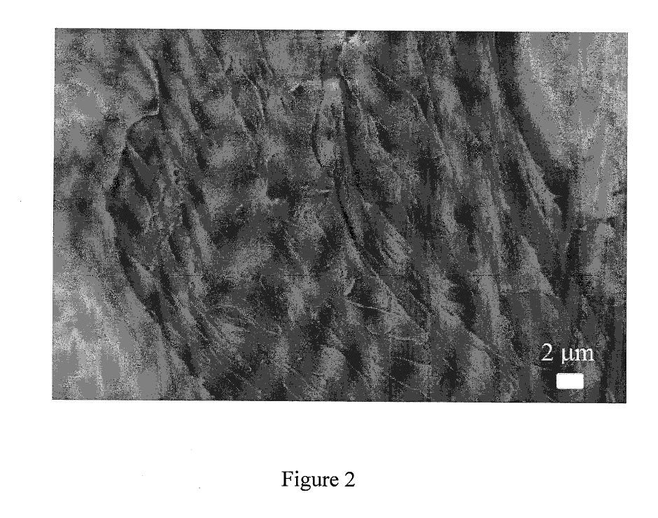

FIGS. 1A, 1B and 2 demonstrate that collagen nanofibers are unstable in an aqueous environment. Using a composition comprising genipin, the Applicants have demonstrated increased stability of electrospun collagen nanofibers in an aqueous environment of both water and Dulbecco's Modified Eagle's Medial (DMEM) for up to three days.

As such, a novel composition is provided useful for promoting the stability of fibers in an aqueous environment, said composition comprising genipin in an amount effective to prevent, ameliorate and/or reduce destabilization of the fibers in the aqueous environment. In one aspect, the composition of the invention comprises no less than about 0.5 wt % of genipin.

Any fiber can be treated using the genipin-based composition of the present invention. Examples of fibers that can be treated with the composition of the present invention include, without limitation: collagen, elastin, chitosan, gelatin, silk, fibrin, laminin, polyamides.

The term “nanofiber” is used generally to refer to a fiber with a diameter less than 1 micron. Nanofibers may be obtained by a number of processes. Three of the most common processes to produce nanofibers include electrospinning, meltblowing and spunbonding. These processes and resulting products share two characteristics: (a) the process begins with a liquid phase polymer and makes fibers and webs directly in a one-step process; and (b) the resulting products comprises polymeric fibers with no other binders, resins or additives (Grafe, T. and Graham K., “Polymeric nanofibers and nanofiber webs: a new class of nonwovens”, Joint INDA-TAPPI Conference, Atlanta, Ga., Sep. 24-26, 2002). In the examples provided herein, the Applicants used electrospinning, however, the present invention is not limited to electrospun fibers. Other methods that can be used to make nanofibers include phase separation, self assembly, especially with collagen and elastin-mimetic polypeptides. For the production of microfibers, the well known wet spinning methods can be used.

Electrospinning is an easy and inexpensive method known in the art of producing long, continuous, polymeric nanofibers. Electrospinning has been applied to both natural and synthetic polymers, including structural proteins such as collagen. These fibers find applications in many industrial and biomedical fields. Of particular interest is the preparation of nanofibrous scaffolds for use in tissue regeneration/engineering of cardiovascular, neural and muscular-skeletal tissues.

Electrospinning uses an electric field to draw a polymer melt or polymer solution from the tip of a capillary to a collector. A voltage is applied to the polymer, which causes a jet of the solution to be drawn toward a grounded collector. The file jets dry to form polymeric fibers, which can be collected on a web. The electrospinning process has been documented using a variety of polymers, including proteinaceous fibers such as collagen. The electrospinning process has been described in U.S. Pat. No. 1,975,504.

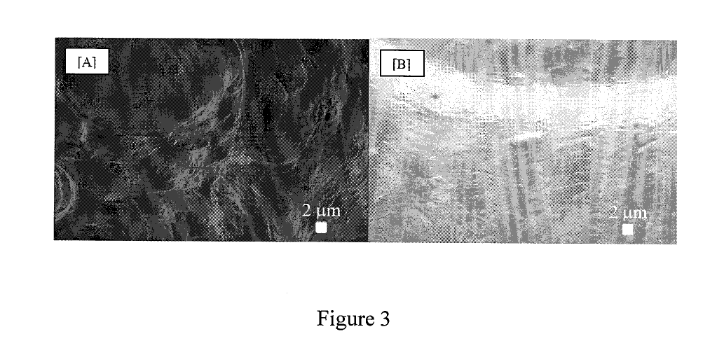

Electrospun collagen fibers, and in particular collagen nanofibers, are unstable in water and genipin is not volatile enough to allow the crosslinking reaction to be carried out in the vapour phase. Thus, the Applicants developed a composition comprising an effective amount of genipin and a solvent system to allow the crosslinking reaction with the genipin-based composition. The solvent system is based on a combination of a solvent, such as alcohol, and water. As shown in FIGS. 3A and 3B, a range of genipin concentrations (0.03-0.1 M) in absolute ethanol or isopropanol failed to maintain the morphology and overall architecture of collagen nanofibers after exposure to water, even after crosslinking for 5 days. However, with the addition of water to the solvent system it was observed that there were certain alcohol/water concentration combinations that maintained the nanofiber morphologies. As a result, the Applicants carried out a systematic study to determine the effect of changing the genipin-based crosslinking solution composition on collagen nanofiber stability in an aqueous environment. The non-limiting combinations of reaction conditions investigated that resulted in good, stable nanofiber formation are presented in Table 1.

Non-limiting examples of solvent systems include methanol/water, propanol/water, n-butanol/water, sec-butanol/water, isobutanol/water and tert-butanol/water. Suitable water content in the solvent system is from about 1 v/v % to about 5 v/v %. However, if the water content is too high, the composition of the present invention may not work well as the fibers would swell before crosslinking becomes effective.

As such, in one aspect of the present invention, a novel composition is provided for cross-linking a polymer fiber, said composition comprising genipin and a solvent system, wherein said solvent system comprises a solvent and water. In one aspect of the present invention, the solvent is an alcohol.

The Applicants discovered that the degree of swelling of fibers treated with the composition of the present invention ranges from a low of 0% for condition 2 of Table 1 to a high of more than 18% for condition 3 of Table 1, after 3 days. This ability to control swelling of the collagen nanofibers has important implications in tissue engineering and other applications.

The degree and rate of swelling of these fibers are associated with their strength and rate of degradation. In a tissue engineering environment, the decrease in strength and the rate of degradation of a collagen scaffold has to be designed such that they are equal to or smaller than the rate of deposition and organization of the extracellular matrix being deposited by the cells to ensure geometric and structural integrity. Since the rate of extracellular matrix production and organization is cell type dependent, it is important that the rate of degradation in the scaffold material be properly designed. There are two main approaches to control degradation rate: (1) by blending two or more polymers with different degradation rates to achieve the desired degradation rate and (2) by controlling the degree of cross-linking. The results presented herein would allow for such control on collagen nanofibrous scaffold by controlling the genipin crosslinking conditions. An example demonstrating the importance of controlling scaffold degradation rate is in the tissue engineering of heart valves. An ideal scaffold in this case would allow for cellular alignment in order to promote collagen alignment similar to the native tissue in order to achieve similar mechanical properties to the native tissue. A rapid degradation rate compared to extracellular matrix (ECM) deposition will inhibit cellular alignment and thus the failure to achieve similar native mechanical properties. Moreover, if the scaffold degrades much slower compared to ECM deposition, then mechanical properties will not be matching those of the native tissue due to the presence of scaffolding material. Therefore, an ideal scaffold should degrade at an equivalent rate of ECM deposition. Other examples include bone, cartilage, artery, nerve and skin regeneration.

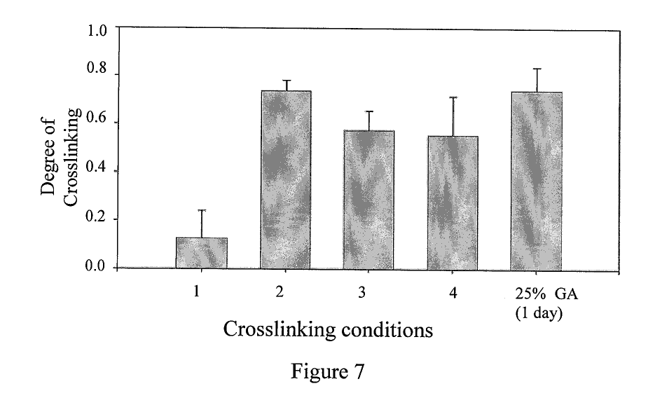

The degree of crosslinking of collagen fibers can be measured using the ninhydrin assay (Chang, W. H., et al., Journal of Biomaterials Science-Polymer Edition 2003, 14, (5), 481-495; Starcher, B. Analytical Biochemistry 2001, 292, (1), 125-129; and Sung, H. W., et al., Journal of Biomedical Materials Research 1999, 47, (2), 116-126). This assay detects the amount of free amino acids in solution by forming a purple complex upon the reaction of ninhydrin with free amino acids. Thus, the more crosslinked the sample, the less free amino acid groups available for the ninhydrin reaction and the lower the purple color intensity determined at a wavelength of 570 nm. FIG. 7 summarizes the degree of crosslinking for the crosslinking conditions of Table 1. A GA-crosslinked sample is included for comparison. As it can be seen in FIG. 7 all crosslinking conditions of Table 1 are effective to varying degrees. It is interesting to note that although glutaraldehyde is quite effective in crosslinking collagen, it is not very effective in controlling its swelling properties (Rho, K. S., et al., Biomaterials 2006, 27, (8), 1452-61).

The instant invention also encompasses therapeutic strategies that involve using fibers cross-linked with the genipin-based composition of the present invention. Collagen and genipin are naturally occurring biodegradable, biocompatible materials that have been investigated for use in a variety of biomedical applications including wound dressings, sutures, tissue engineering and drug delivery. In one aspect, fibers cross-linked with the genipin-based composition of the present invention may be used in the manufacture of a drug delivery composition for the controlled release of a pharmaceutically active agent. In another aspect, fibers cross-linked with the genipin-based composition of the present invention may be used in a method for treating skin or lip related anomalies.

In another aspect, the present invention also relates to methods for modulating the rate of release of a bioactive compound from a device for pharmaceutically active agents comprising a pharmaceutically active agent incorporated within or between polymeric fibers treated with the genipin-based composition of the invention. By “modulate” or “modulating”, it is meant that the rate or release of the bioactive compound incorporated within of between the polymeric fibers of the delivery system is increased or decreased. Methods for modulating the rate of release include increasing or decreasing loading of the pharmaceutically active agent incorporated within or between the fibers treated in the genipin composition of the invention, selecting polymers to produce the polymeric fibers which degrade at varying rates, varying polymeric concentration of the polymeric fibers and/or varying diameter of the polymeric fibers. Varying one or more of these parameters can be performed routinely by those of skill in the art based upon teachings provided herein. A list of pharmaceutically active agents that can be modulated in accordance with the present invention include: silver nanoparticles (for wound healing applications), growth factors (to control cell proliferation and differentiation in tissue engineering applications), genes (for gene delivery applications), anti-cancer agents, such as paclitaxel, and anticoagulants (drug eluting stents).

Genipin as a chemical crosslinking agent possesses low cytotoxicity and is more stimulative to cell proliferation compared to glutaraldehyde, currently the most popular crosslinking agent used to stabilize electrospun collagen and other protein fibers. The novel compositions and methods of the present invention, when coupled with the recently developed method for the creation of various 3D macrostructures from electrospun nanofibers, will provide a broad range of structure for tissue engineering and other applications (Zhang, D. and Chang, J. Nano Lett 2008, 8, (10), 3283-7).

### iii. Exemplification

The following non-limiting examples are illustrative of the present invention.

### Example 1

### Preparation of Collagen Fibers

Materials

Rat tail collagen type 1 was purchased from Sigma Aldrich (C7661); 1,1,1,3,3,3 Hexafluoroisopropanol (≧99%) was purchased from Sigma Aldrich (105228); Glutaraldehyde (25% in water) was purchased from Sigma Aldrich (G5882); Dulbecco's Modified Eagle Medium (DMEM) was purchased from Invitrogen (12571-063); Anhydrous Isopropanol (99.7%) was purchased from Caledon labs (8601-2); Genipin was purchased from Challenge Bio Products Ltd.

Determination of Collagen Fiber Diameters and Calculating Fiber Swelling

All samples were imaged using a Scanning Electron Microscope (Leo 1530) and diameters of 100 randomly selected fibers were measured, per sample, using image processing software (ImageJ). One-way ANOVA using the Tukey test was used to compare the difference between the diameters of crosslinked samples (D_{crosslink}) and after exposure to growth media for 1 and 3 days (D_{final}). If a significant difference existed, the percent swelling was then calculated using the equation:

$$\frac{{D}_{final} - {D}_{crosslink}}{{D}_{crosslink}} \times 100$$

Measuring the Degree of Crosslinking Using the Ninhydrin Assay

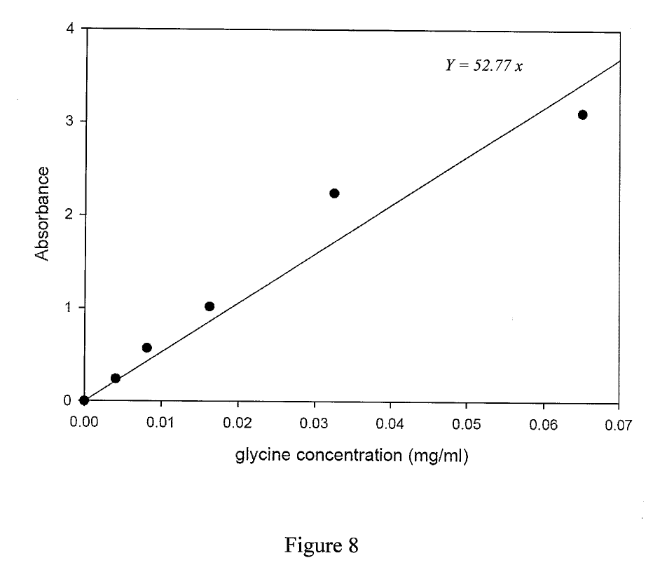

The results are expressed as a ratio with reference to that of the uncrosslinked sample. First, the samples were dried, weighed (Wₛₐₘₚₗₑ) and then placed in vials containing 1 ml of ninhydrin solution and 2 ml of distilled water; the samples were then heated at 80° C. for 15 minutes. The supernatant was then removed and the absorbance at 570 nm was measured for each sample. To translate the absorbance measurement into amine concentration, a calibration curve was constructed using glycine solutions of a range of concentrations (0.0-0.7 mg/ml) (FIG. 8). The calibration curve was used to translate the absorbance into amino acid concentration. The mass of free amino acids (W_{free}) was calculated by multiplying the amino acid concentration by the total volume (3 ml). The ratio of free amino acids to initial mass was then calculated for each group R=W_{free}/Wₛₐₘₚₗₑ. The degree of crosslinking was then calculated using: 1−R_{crosslink}/Rₐₛ₋ₛₚᵤₙ.

Collagen Electrospinning

The collagen type 1 from rat-tail was electrospun from a 5 wt % collagen in a 1,1,1,3,3,3 hexafluoroisopropanol solution. The electrospinning equipment consists of a high voltage power supply, a metal plate collector connected to the high voltage power supply, and a syringe pump placed on a mechanical jack for position control. A 1 ml plastic syringe and a blunt-ended 18.5-gauge stainless steel needle were used to introduce the collagen solution into the electric field. A metal electrode was attached to the needle to serve as the ground. The electrospinning parameters used were: voltage of 22 KV, flow rate of 0.2 ml/hr and a tip to collector distance of 13 cm. Fibers were electrospun onto the collector plate.

### Example 2

### Effect of Changing Crosslinking Solution Composition on Collagen Fiber Stability

The experimental parameters investigated were: solvent (isopropanol, ethanol), water content (0%, 1%, 3% and 5%) and reaction time (1, 3 and 5 days). All electrospun collagen fiber samples were exposed to air and the reaction temperature was maintained at 37° C. in an incubator. The genipin concentration was fixed at 11.3 mg of genipin per mg of collagen, which was sufficient for the crosslinking reaction to reach completion (Yao, C. H., et al., Materials Chemistry and Physics 2004, 83, (2-3), 204-208). After crosslinking, the collagen samples were washed in ethanol or isopropanol (depending on the solvent used) for further characterization.

TABLE 1
Crosslinking conditions for electrospun collagen nanofibers with
genipin that yielded stable fibers after exposure to an aqueous
environment

| | | Water | Crosslinking |
|---|---|---|---|
| Crosslinking | | content | time |
| condition | Solvent | (v/v %) | (days) |
| 1 | Ethanol | 5 | 3 |
| 2 | Ethanol | 3 | 5 |
| 3 | Ethanol | 5 | 5 |
| 4 | Isopropanol | 5 | 5 |

Results

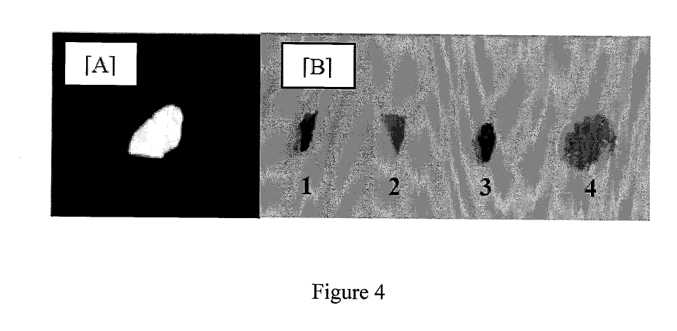

The genipin crosslinking reaction is associated with a color change which can be easily visualized. As the reaction progresses, a greenish color develops initially and eventually becomes blue (Butler, M. F., et al., Journal of Polymer Science Part a-Polymer Chemistry 2003, 41, (24), 3941-3953). The color difference between the as-spun sample (white) and the genipin-crosslinked samples can be observed in FIG. 4. Samples 1 and 3 have a deep blue color as compared to samples 2 and 4, which are green. It is important to mention however, that all samples turn deep blue after exposure to water; this illustrates the importance of water in the blue color formation. Although there have been several studies on the mechanism of the crosslinking reaction, its relationship to the blue color formation is still unknown (Touyama, R., et al., Chemical & Pharmaceutical Bulletin 1994, 42, (8), 1571-1578; Touyama, R., et al., Chemical & Pharmaceutical Bulletin 1994, 42, (3), 668-673; and Butler, M. F., et al., Journal of Polymer Science Part a-Polymer Chemistry 2003, 41, (24), 3941-3953).

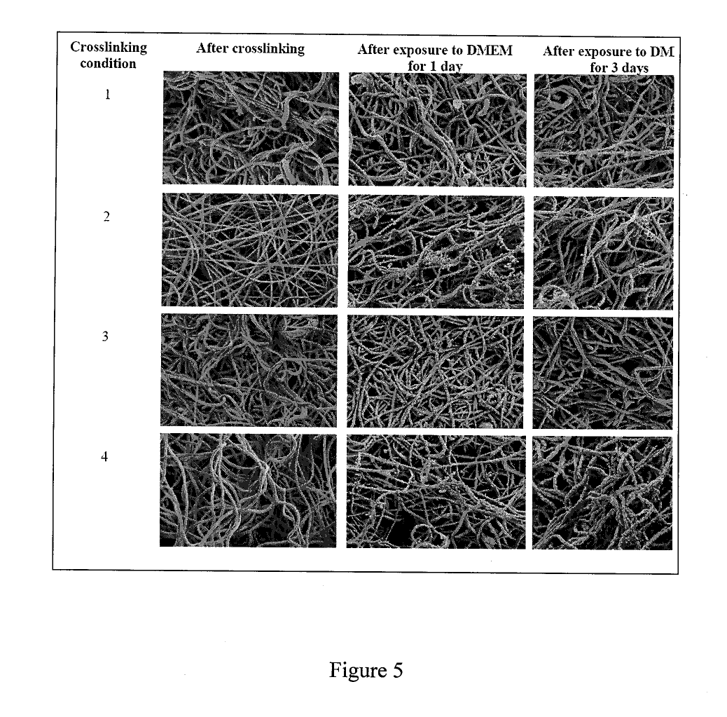

FIG. 5 shows scanning electron microscope (SEM) images of the collagen fibers morphologies crosslinked using the four conditions listed in Table 1, before and after immersion into DMEM for up to 3 days. All fibers remain intact, although for those samples exposed to DMEM, salt deposits from the media onto the fibers can be observed. The DMEM growth media contains an appreciable amount of salt and due to their low solubility in alcohol they cannot be removed completely after washing. FIG. 5 illustrates that not only the polymer fiber morphology is maintained, but also the degree of swelling among all samples is minimal. These results can be contrasted with those reported based on GA vapor crosslinking of collagen fibers, wherein the collagen fibers showed significant swelling and the formation of gel-like structures. GA vapour failed to maintain collagen fiber morphology and led to a reduction in porosity in the samples. For applications such as tissue engineering scaffolds this change could be significant, since high porosity and pore interconnectivity of the non-woven structure are essential for cell migration and proliferation in the 3D structure. Therefore, it is important that the crosslinked collagen fibers not only stay intact, but also exhibit swelling control.

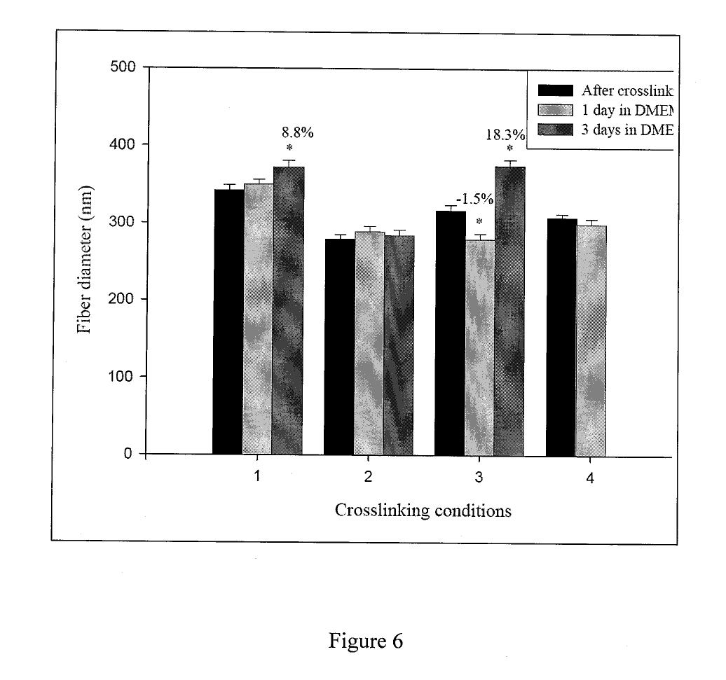

The degree of swelling among all samples is minimal. The degree of swelling of the genipin crosslinked fibers is quantified in terms of the change in average fiber diameters and the percent swelling are presented in FIG. 6. Swelling was significant after 3 days in both crosslinking conditions 1 and 3. Crosslinking condition 2 however, did not exhibit any swelling after 3 days in DMEM, while crosslinking condition 4 resulted in non-uniform fiber diameter which was probably due to either fiber degradation or selective regional swelling of the fiber in DMEM, and it was not possible to determine the fiber diameters accurately. In this case the degree of swelling is probably the highest among the crosslinking conditions investigated.

FIG. 7 summarized the results for all crosslinking conditions listed in Table 1. The GA-crosslinked sample is included for comparison.

FIG. 7 shows that all crosslinking conditions of Table 1 are effective to varying degrees. Reaction condition 1 gives the lowest degree of crosslinking, while all other conditions give significantly higher results. The highest degree of crosslinking is for samples treated with condition 2, which collaborates well with the lowest average fiber diameter change shown in FIG. 6 for up to 3 days in DMEM. Several trends are also apparent. A comparison of the crosslinking conditions 1 and 3 reveal that in ethanol, increasing reaction time (3 to 5 days) at constant water content (5%) increases the degree of crosslinking. Also the degree of crosslinking in ethanol and isopropanol are comparable (conditions 3 and 4) although the morphological changes upon exposure to DMEM are quite dissimilar (FIG. 5). It is interesting to note that although glutaraldehyde is quite effective in crosslinking collagen, it is not very effective in controlling its swelling properties (Rho, K. S., et al., Biomaterials 2006, 27, (8), 1452-61).

The non-limiting results presented herein, demonstrate that electrospun collagen nanofibers can be stabilized in an aqueous environment by using the novel composition comprising genipin in an alcohol-water mixed solvent system. Moreover, the degree of swelling of the fiber can also be controlled. Such control is important if these fibers are used to form non-woven scaffolds for tissue engineering applications. Initial stability and geometry control of these fibers are important since structural integrity, porosity and pore connectivity maintenance are critical at least at the early stages of tissue engineering.

### iv. Equivalents

While the present invention has been described with reference to what are presently considered to be preferred examples, it is to be understood that the invention is not limited to the disclosed examples. To the contrary, the invention is intended to cover various modifications and equivalent arrangements included within the spirit and scope of the appended claims.

### v. Incorporation by Reference

All publications, patents and patent applications cited are herein incorporated by reference in their entirety to the same extent as if each individual publication, patent or patent application was specifically and individually indicated to be incorporated by reference on its entirety.

We claim:

1. A composition for cross-linking fibers, characterized in that said composition comprises genipin.

2. A composition useful for promoting the stabilization of fibers in an aqueous environment, characterized in that said composition comprises genipin in an amount effective to prevent, ameliorate and/or reduce destabilization of the fibers in the aqueous environment.

3. The composition according to any one of claims 1 and 2, characterized in that said composition further comprises a solvent system.

4. The composition of claim 3, characterized in that the solvent system comprises a solvent and water.

5. The composition of claim 4, characterized in that the water in the solvent system is present in an amount from about 1 v/v % to about 5 v/v % of the total solvent.

6. The composition of claim 4, characterized in that the solvent is an alcohol.

7. The composition of claim 6, characterized in that the alcohol in the solvent system is selected from the group consisting of ethanol and isopropanol.

8. The composition according to any one of claims 1 and 2, characterized in that the composition comprises no less than about 0.5 wt % of genipin.

9. The composition according to any one of claims 1 and 2, characterized in that each fiber comprises a continuous nanofiber.

10. The composition according to any one of claims 1 and 2, characterized in that the fibers are selected from the group comprising of: collagen, elastin, aminopolysaccharides, gelatin, silk, fibrin, laminin and polyamides.

11. The composition according to any one of claims 1 and 2, characterized in that the aqueous environment comprises an extra-cellular matrix.

12. A composition for cross-linking continuous nanofibers, characterized in that said composition comprises genipin, an alcohol solvent and water, wherein said genipin, alcohol, and water are provided in an amount effective to prevent, ameliorate and/or reduce destabilization of the continuous nanofibers in an aqueous environment.

13. A method of cross-linking fibers, characterized in that said method comprises the step of contacting the fibers with a composition comprising genipin.

14. A method of promoting the stabilization of fibers in an aqueous environment, characterized in that said method comprises the step of contacting the fibers with a composition comprising genipin in an amount effective to prevent, ameliorate and/or reduce destabilization of the fiber in the aqueous environment.

15. A method of controlling the degree of swelling of a fiber in an aqueous environment, characterized in that said method comprises contacting the fiber with a composition comprising genipin, an alcohol and water for a time of treatment, wherein the degree of swelling is controlled by selecting the amounts of genipin, alcohol or water in the composition, or by selecting the time of treatment.

16. The method according to any one of claims 13 to 15, characterized in that said composition further comprises a solvent system.

17. The method of claim 16, characterized in that the solvent system comprises a solvent and water.

18. The method of claim 16, characterized in that the water in the solvent system is present in an amount from about 1 v/v % to about 5 v/v % of the total solvent.

19. The method of claim 17, characterized in that the solvent is an alcohol.

20. The method of claim 19, characterized in that the alcohol in the solvent system is selected from the group consisting of ethanol and isopropanol.

21. The method according to any one of claims 13 to 15, characterized in that the composition comprises no less than about 0.5 wt % of genipin

22. The method according to any one of claims 13 to 15, characterized in that each fiber comprises a continuous nanofiber.

23. The method according to any one of claims 13 to 15, characterized in that the fibers are selected from the group comprising of: collagen, elastin, aminopolysaccharides, gelatin, silk, fibrin, laminin and polyamides.

24. The method according to any one of claims 13 to 15, characterized in that the aqueous environment comprises an extra-cellular matrix.

25. A fiber, characterized in that said fiber has been treated in any of a composition comprising genipin in an amount effective to prevent, ameliorate and/or reduce destabilization of the fiber in an aqueous solution.

26. A scaffold, characterized in that the scaffold comprises fibers treated in a composition comprising genipin in an amount effective to prevent, ameliorate and/or reduce destabilization of the fibers in an aqueous environment. In aspects of the invention, the scaffold further comprises at least one cell.

27. A method of preparing nanofibrous scaffolds for use in tissue regeneration/engineering, said method comprising the following steps: (a) producing nanofibers; (b) treating the nanofibers with a composition comprising genipin, alcohol and water, and wherein said genipin, alcohol solvent and water are present in an amount effective to prevent, ameliorate and/or reduce destabilization of the nanofibers in an aqueous environment.

28. A method of treating a dermatological condition comprising the step of topically applying to the skin or lip a collagen fiber treated with a composition comprising genipin, alcohol and water, wherein said genipin, alcohol and water are present in an amount effective to prevent, ameliorate and/or reduce destabilization of the collagen fiber in an aqueous environment.

29. A device for the controlled release of a pharmaceutically active agent, said device comprising: (a) a fiber matrix, wherein the fiber in the matrix includes a primary amine group and the polymer fiber is treated with a composition comprising genipin, alcohol and water, wherein said genipin, alcohol and water are present in an amount effective to prevent, ameliorate and/or reduce destabilization of the collagen fiber in an aqueous environment; and (b) a pharmaceutically active agent, wherein said pharmaceutically active agent is incorporated in the polymer fiber matrix.
